(19) United States
(12) Patent Application Publication
(10) Pub. No.: US 2012/0116457 A1
(43) Pub. Date: May 10, 2012

(54) Stabilizer for assisting stabilization of a spinal implant and method of using the stabilizer

(75) Inventor: Frank Castro, Louisville, KY (US)

(73) Assignees: Limited Liability Company;; Cardinal Spine, LLC

(21) Appl. No.: 12/927,050

(22) Filed: Nov. 6, 2010

(51) Int. Cl. A61B 17/88; A61B 17/70

(52) U.S. Cl. 606/279; 606/246

(57) ABSTRACT

A stabilizer for receiving an end of a spinal implant. The stabilizer includes a gap that creates a socket for receiving the spinal implant. Bodies of the stabilizer are manufactured in a plurality of shapes. A cover is capable of blocking egress of the spinal implant from the socket.

### BACKGROUND OF THE INVENTION

1. Field of the Invention

Among other things, the present invention is related to a stabilizer for a spinal implant. Preferred embodiments of the current invention have a gap for creating a socket for receiving a lengthwise end of the spinal implant. In the practice of the current invention, the stabilizer's body is fastened to bone. The stabilizer's cover is attachable to the body and blocks egress of the spinal implant from the socket as well egress of the fasteners securing the stabilizer's body to bone. Preferred embodiments of stabilizers are manufactured in many geometric shapes and can be utilized with a plethora of spinal implants.

2. Description of the Previous Art

Any discussion of references cited in this Description of the Previous Art merely summarizes the disclosures of the cited references and Applicant makes no admission that any cited reference or portion thereof is relevant prior art. Applicant reserves the right to challenge the accuracy, relevancy and veracity of the cited references.

1) U.S. Pat. No. 4,961,740-Ray, et al. enables a V-thread fusion cage and method of fusing a bone joint. In part, Column 6 of Ray reads, “The fusion basket 10 of FIG. 1 was formed from a solid steel cylinder by drilling eight small, equally spaced holes 11 in the axial direction, each hole being centered on a circle concentric with the axis of the cylinder. Then a large hole was drilled centered on the axis and having a radius substantially identical to that of the aforementioned circle. A V-thread 12 was then machined in the external surface of the cylinder, thus opening through that surface a perforation 13 extending through the rounded valley 14 of the V-thread at each crossing of the valley and one of the small holes 11. A screw thread 15 was then machined in the internal surface of the fusion basket to threadably receive an end cap 16 that has apertures 18 similar to those of a salt shaker. Snap-on end caps would also be useful.”

Among other things, U.S. Pat. No. 4,961,740 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

2) U.S. Pat. No. 6,344,057-Rabbe, et al. enables an adjustable vertebral body replacement. In part, Column 6 of Rabbe reads, “In one important feature of the cylindrical body 21, the opposite ends of the cylindrical wall 25 are formed into external threads 32. In one specific embodiment, the threads 32 extend from each opposite end over most of the total length of the threaded cylindrical body 21 and are configured to engage the threaded endplates 22. Each endplate includes a flange 35, which preferably assumes a shape to cover a substantial load-bearing area of the endplates of the adjacent intact vertebral bodies. A cylinder 37 is integrally formed with flange 35 to extend toward the threaded cylindrical body 21 when the endplates 22 are placed within the excised vertebral space. The cylinder 37 of each endplate includes a number of threaded openings 39 adapted to receive a set screw 24 therein.”

Among other things, U.S. Pat. No. 6,344,057 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

3) U.S. Pat. No. 6,582,432-Michelson enables a cap for use with artificial spinal fusion implant. In part, Column 8 of Michelson reads, “The open end 54 of the cylindrical implant 50 has an internal thread 51 for receiving a complementary cap 52 which has an external thread 58 for engaging the internal threads 51 of the cylindrical member 50. As shown in FIG. 5, cap 52 has an exposed exterior surface opposite an unexposed interior surface with a mid-longitudinal axis passing through the exterior and interior surface of the cap. A cross section of cap 52 along a plane parallel to the mid-longitudinal axis is at least in part convex along the exterior surface of cap 52. A second cross-section of cap 52 along a second plane parallel to the mid-longitudinal axis of cap 52 is curved along at least a portion of the exterior surface of cap 52 so that the curvature of the first and second cross-sections form a portion of a sphere. As shown in FIG. 4 a, cap 52 has rounded edges in a plane transverse the mid-longitudinal axis of cap 52. The cap 52 has a hexagonal opening 59 for use with an allen wrench for tightening the cap.”

Among other things, U.S. Pat. No. 6,582,432 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

4) U.S. Pat. No. 6,783,547-Castro enables an apparatus for fusing adjacent bone structures. In part, Column 5 of Castro reads, “With reference to FIGS. 13-15, apparatus 10 may further include end caps 60 which are mounted to one or both of the exposed longitudinal ends (depending whether outer cage or inner cage is provided with an end face) of the inner and outer cages 12, 14. Each end cap 60 includes peripheral collar 62 and insertion portion 64 depending from the collar 62. Collar 62 defines an enlarged cross-section relative to the longitudinal ends of inner and outer cages 12, 14 to engage the respective ends of the cages 12, 14. Insertion portion 64 includes a plurality of arcuate internal springs 66 depending axially from the collar 62. Internal springs 66 are adapted to flex inwardly upon positioning of end cap 60 within the respective cage, but, return outwardly under the influence of their resilient characteristics, to engage the inner surfaces of the inner or outer cages 12, 14, thereby securing the end cap 60 to the respective cage 12, 14. Arcuate springs 66 may be connected to collar 62 by conventional means and are preferably fabricated from a resilient plastic or metallic material. End cap 60 further defines central opening 68. End cap 60 is shown circular in cross-section for use with a circular inner and outer cage 12, 14 although it is appreciated that end cap 60 may be elliptical if desired. A plurality of spaced individual cone-shaped spikes 70 extend from collar 62 for penetrating the vertebral end plates when the apparatus is positioned within the intervertebral space.”

Among other things, U.S. Pat. No. 6,783,547 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

5) U.S. Pat. No. 7,235,105-Jackson enables a threaded center line cage with winged end gap [cap] (sic). In part, Column 6 of Jackson reads, “Referring to FIGS. 1 and 13-16, the end cap 3 includes a center section 30 and wing sections 32 extending laterally of the center section 30 and curving in a posterior direction therefrom. The front of the end cap 3 is preferably sized, shaped and designed to follow the contour of the front or anterior edge of the vertebrae 6 and 7. The end cap 3 includes structure for securing it to the spacer member 2. The illustrated end cap 3 includes a pair of opposed resilient pawls 34 extending from a posterior surface 36 (FIG. 13) of the end cap 3 at the center section 30. The pawls 34 are positioned to engage recesses 38 (FIGS. 1 and 14) formed into the lateral surfaces 12 of the spacer member 2 by deforming as the end cap is slid over the anterior end of the spacer member (see FIG. 14) and then resiliently returning to a gripping shape (as seen in FIG. 15) to hold the end cap 3 on the spacer number 2. Alternatively, other structure or means for securing the end cap 3 to the spacer member 2 may be employed in the assembly 1.”

Among other things, U.S. Pat. No. 7,235,105 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

6) US Pub. Patent Application 20030083746-Kuslich discloses a vertebral spacer for spinal stabilization. Paragraphs 63 and 64 of Kuslich read, 10063] End caps 70 and 72 are comprised of an end cap body 74 and include an engagement surface 76. The engagement surface 76 may be defined by an engagement lip 78 such as is shown in FIG. 15 or alternatively as a pair of engagement members 80 and 82 which define a groove 84 such as is shown in FIGS. 10, 12 and 16. Other configurations of engagement surfaces may be utilized. [0064] As may be seen in FIG. 14, end caps 70 and 72 may be configured to have a variety of shapes to allow the second engagement surface 86 of the end cap to engage the surface 56 and 58 of a spinal body 52 and 54, regardless of the relative angle between the device 10 and spinal body 52 or 54. In addition, the end caps 70 and 72 may be provided in various sizes to allow a body 12 of a standard size and shape to be used in a wide variety of sizes of intervertebral spaces 60. In the various embodiments shown herein the end caps 70 and 72 may have a diameter equal to or larger than the diameter 100 (illustrated in FIG. 1) of the body 12.”

Among other things, US Published Patent Application 20030083746 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

7) US Pub. Patent Application 20040088054-Berry discloses a laterally expandable cage. Paragraph 22 of Berry reads, “[0022] A laterally expandable spinal implant 100 according to one embodiment of the present invention will now be described with reference to FIGS. 1-6. As shown in FIGS. 1 and 2, the implant 100 includes a central member or cage 102, a pair of lateral members or wings 104 that are adapted to laterally extend from the cage 102, and an expansion mechanism 106 (or means) that is operable to extend the wings 104. In the illustrated embodiment, the expansion mechanism 106 includes a turnbuckle or threaded shaft 108 that connects the wings 104 together. In other embodiments, the expansion mechanism can include hydraulic pistons, mechanical linkages, and the like. The shaft 108 includes a gear 110 that is centrally located on the shaft 108 between opposing threaded portions 112 and 114. In one embodiment, threads 116 on the threaded portions 112 and 114 are oppositely threaded (i.e., one is a left handed thread and the other is a right handed thread.) In one form of the present invention, the threads 116 of the threaded portions 112 and 114 have an equal pitch such that the wings 104 are able to extend from the central member 102 at the same rate. This ensures that the implant 100 has a symmetrical configuration, which in turn aids in centering the implant 100 over the vertebrae. The threaded portions 112 and 114 threadedly engage threaded openings 118 that are defined in each of the wings 104. In another embodiment, only one end of the shaft 108 is threaded, while the other end of the shaft 108 is unthreaded. With this embodiment, the wings 104 are still extended by rotating the shaft 108.”

Among other things, US Published Patent Application 20040088054 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

8) US Pub. Patent Application 20040122518-Rhoda discloses an intervertebral implant. Paragraph 105 of Rhoda reads, “[0105] As shown in FIGS. 17-20, upper endcap 402 also includes two elongated bores 410 which can be filled with bone growth inducing substances to allow bony ingrowth and to further assist in the fusion of the adjacent vertebrae. Upper endcap 402 further includes a central bore 411 for receiving a fastening member, such as a screw. In addition, upper endcap 402, on its upper surface 405, has sections or areas having teeth 412 or similar gripping means to facilitate engagement of implant 400 with the end plates of the adjacent vertebra, and has sections or areas 414, 416 which are substantially smooth and devoid of any protrusions. Although in FIG. 17 sections 414, 416 are shown as extending along the entire length of upper endcap 402, from perimeter edge to perimeter edge, sections 414, 416 may extend only partially along the length of upper endcap 402. Sections 414, 416 are provided to assist the surgeon in anterior or lateral implantation of the implant as was discussed above with respect to sections 22, 24. As can be seen in FIGS. 18 and 21, upper endcap 402 has a generally rectangular protrusion 418 configured and dimensioned to interface and mate with a recess portion of the implant body or with the lower endcap. While protrusion 418 has been shown and described as generally rectangular, it can be appreciated that protrusion 418 can be any shape desired. A lower surface 407 surrounds the protrusion 418. Lower surface 407 is illustrated as surrounding and encircling completely protrusion 418, but it can be appreciated that lower surface 407 may only partially surround protrusion 418.”

Among other things, US Published Patent Application 20040122518 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

9) US Pub. Patent Application 20040225360-Malone discloses devices and methods for facilitating controlled bone growth or repair. Paragraph 72, in part, of Malone reads, “Ends 24, 26 of the cage body 22 are provided with a non-perforated closure. In the illustrated embodiment, the anterior end 26 is closed by an integral non-perforated end wall 46, while there is provided a removable end cap 48 securable, by threaded attachment, friction fit or otherwise, to the posterior end 24 of the cage body 22. The end cap 48 may be provided with a recess 50 for receiving an insertion tool, for example if the end cap is made to threadably connect to the cage body, and there is preferably provided on the top of the end cap 48 a line score 52 for aiding proper orientation of the device in the vertebral interspace.”

Among other things, US Published Patent Application 20040225360 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

10) US Pub. Patent Application 20080058939-Hughes, et al. discloses a revision spacer. Paragraph 20 of Hughes reads, “The upper endplate module 32 includes an outer surface 38 and an inner surface 40 with passages 42 extending through the module 32 including through the outer and inner surfaces 38, 40. A keel 44 extends from the outer surface 38 and is adapted to engage the vertebral body 12 when the device 30 is inserted into the disc space 20. The upper endplate module 32 may further include access ports 45 to permit manipulation of the endplate module with an insertion or extraction tool. The ports 45 may also allow for eventual bone ingrowth. The upper endplate module 32 may also include one or more radiolucent markers 47 for monitoring the position of the device 30 during and after implantation using fluoroscopy.

Among other things, US Published Patent Application 20080058939 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

11) US Pub. Patent Application 20090138083-Biyani discloses a variable height vertebral body replacement implant. Paragraph 29 of Biyani reads, “Depending upon the anatomy of the patient, it maybe desirable to also provide a wedge-shaped end cap 124 on one or both of the end rings 115, 119. The respective end caps 124 each has a distal end surface 125 disposed at an angle A (see FIG. 8) in the range of 2° to 10° with respect to a plane P perpendicular to the axis X centered between the joined insertion rod sections 117 and hollow members 120. The end caps 124 can be fastened to the respective end rings 115, 119 by any desired fastening means. Each of the end caps 124 has a plurality of serrations or projections 128 extending from its distal end surface 125.”

Among other things, US Published Patent Application 20090138083 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

12) US Pub. Patent Application 20090187245-Steiner, et al. discloses an interbody fusion hybrid graft. Paragraph 35 of Steiner reads, “The composite cortical bone block body or intervertebral spacer 10 is preferably constructed with a first end cap member 12 constructed of cortical bone taken from donors cut into a ring shape. The cap member body 13 has an interior circular through going bore 14 formed or cut therein, and defines a flat planar bottom surface 16 which is provided with a dovetail shaped projection 18 which extends outward from the planar bottom surface 16. The cap body is tapered with the rear end 17 being of a greater height than the front end 19. The outer or top surface 20 which is tapered has a plurality of teeth 22 formed or cut into the exterior surface to provide a gripping surface on the adjacent vertebrae. The taper runs between 5° to 10° and the height of the upper cap member runs between 3-4 mm. The side wall of the ring body is formed with a channel or groove 24. The cortical cap members 12 and 112 have superior wall strength for support between load bearing body structures such as vertebrae. While it is noted that the bottom wall surfaces and are planar, these surfaces can be provided with any kind of complementary construction.”

Among other things, US Published Patent Application 20090187245 does not appear to disclose a socket for assisting stabilization of a spinal implant where the socket comprises a polygonal, cylindrical or elliptic cylindrical body surrounding a cavity, where the body comprises a gap at a first end that creates the socket, an extension that extends beyond a second end for receiving fasteners and a cover attachable to the extension for blocking egress of the spinal implant from the socket.

### SUMMARY OF THE INVENTION

The present invention provides a stabilizer capable of assisting the stabilization of the spinal implant after the spinal implant is inserted into a surgically created cavity. Preferred embodiments of the current stabilizer include a body that is secured to bone. Within the scope of the present invention, the body includes a gap that creates a socket for receiving a lengthwise edge of the spinal implant. In accord with the present invention, sockets can be manufactured in different shapes to accommodate the different shapes of the spinal implants. And a cover is attachable to the body to block egress, among other things, of the spinal implant from the socket.

An aspect of a preferred embodiment of the present invention is to provide a stabilizer for assisting stabilization of a spinal implant.

Still another aspect of a preferred embodiment of the present invention is to provide a stabilizer including a gap therein for creating a socket for receiving a coupling end of the spinal implant.

It is another aspect of a preferred embodiment of the present invention to provide a stabilizer with a polygonal shaped body capable of stabilizing amorphous or geometrically defined spinal implant supporting structures.

Yet another aspect of preferred embodiments the present invention is to provide stabilizers with cylindrical or elliptic cylindrical bodies capable of stabilizing amorphous or geometrically defined spinal implant supporting structures.

Yet still another aspect of preferred embodiments of the present invention is to provide stabilizers with rectangular, trapezoidal and/or hexagonal bodies capable of stabilizing amorphous or geometrically defined spinal implant supporting structures.

It is still another aspect of a preferred embodiment of the present invention to provide a body including an extension that has a plurality of apertures.

Still another aspect of a preferred embodiment of the present invention is to provide a cover including an aperture for aligning with one of the plurality of apertures located in the body's extension.

It is another aspect of the present invention to provide an embodiment that utilizes a fastener to secure the cover to the body.

An embodiment of the present invention can be described as a stabilizer for assisting stabilization of a spinal implant; the stabilizer comprising: a) a polygonal body comprising: i) a perimeter distal from the spinal implant enclosing an opening of the polygonal body; ii) posterior and lateral walls extending from the perimeter toward the spinal implant, wherein the posterior and lateral walls are of approximately identical length; and iii) an anterior wall comprising a first section extending from the perimeter toward the spinal implant and a second section extending from the perimeter away from the spinal implant, wherein: A) the first section is of lesser length than the posterior and lateral walls, thereby creating a socket for fitting about a lengthwise end of the spinal implant; and B) the second section comprises a plurality of apertures for receiving fasteners; b) a cover attachable to the second section capable of: i) blocking fastener egress from a majority of the plurality of apertures; and ii) blocking egress of the spinal implant from the socket; and c) one or more fasteners for securing the cover to the second section.

Another embodiment of the present invention can be described as a stabilizer for assisting stabilization of a spinal implant; the stabilizer comprising: a) a cylindrical or an elliptic cylindrical wall encircling a lumen; b) an opening at each lengthwise end of the wall; c) a rim distal from the spinal implant; d) a gap in the wall at an end of the wall opposite the distal rim creating a socket for fitting about the lengthwise end of the spinal implant; e) an extension proximate the gap and extending beyond the distal rim in a direction away from the spinal implant; the extension comprising a plurality of apertures for receiving fasteners; f) a cover, including a plurality of apertures, attachable to the extension and capable of: i) blocking fastener egress from a majority of the plurality of apertures; and ii) blocking egress of the spinal implant from the socket; and g) one or more fasteners for securing the cover to the second section.

Yet another embodiment of the present invention can be described as a socket for assisting stabilization of a spinal implant; the socket comprising: a) a polygonal, cylindrical or elliptic cylindrical body surrounding a cavity; the body comprising: i) a first end for coupling with an end of the spinal implant, wherein the first end further comprises a gap for receiving the coupling end of the spinal implant into the cavity of the socket; ii) a second end opposite the first end; and iii) an extension extending beyond the second end, wherein the extension comprises a plurality of apertures for receiving fasteners; and b) a cover attachable to the extension capable of: i) blocking fastener egress from a majority of the plurality of apertures; and ii) blocking egress of the spinal implant from the socket.

In still another embodiment, the present invention can be described as a method of assisting with stabilization of a spinal implant comprising the steps of: a) providing a socket for receiving a coupling end of the spinal implant, wherein the socket comprises: i) a polygonal, cylindrical or elliptic cylindrical body surrounding a cavity; ii) a first end comprising a gap for receiving the coupling end; iii) a second end opposite the first end; and iv) an extension extending beyond the second end, wherein the extension comprises a plurality of apertures for receiving fasteners; b) anchoring the socket to bone; and c) attaching a cover to the extension, wherein after attachment to the extension, the cover is capable of: i) blocking fastener egress from a majority of the plurality of apertures; and ii) blocking egress of the spinal implant from the socket.

It is the novel and unique interaction of these simple elements which creates the apparatus and methods, within the ambit of the present invention. Pursuant to Title 35 of the United States Code, descriptions of preferred embodiments follow. However, it is to be understood that the best mode descriptions do not limit the scope of the present invention.

### BRIEF DESCRIPTION OF THE DRAWINGS

FIG. 1 is an exploded view frontal perspective of a preferred embodiment of the stabilizer (20) having a polygonal body (30) and attachable cover (60).

FIG. 2 is a frontal view a preferred embodiment of stabilizer (20) where cover (60) is attached to anterior wall (42) of polygonal body (30).

FIG. 3 portrays a rectangular polygonal body embodiment within the scope of the present invention.

FIG. 3A portrays a nebulous rectangular polygonal body embodiment within the scope of the present invention.

FIG. 4 shows a trapezoidal polygonal body embodiment within the scope of the current invention.

FIG. 4A shows a nebulous trapezoidal polygonal body embodiment within the ambit of the current invention.

FIG. 5 enables a hexagonal polygonal body embodiment within the scope of the present invention.

FIG. 5A enables a nebulous hexagonal polygonal body embodiment within the ambit of the present invention.

FIG. 6 portrays a preferred embodiment of a cylindrical or elliptic cylindrical stabilizer (80).

FIG. 7 is a frontal perspective of stabilizer (80) where cover (60) is attached to extension (102).

FIG. 8 is a top plan view of a preferred cylindrical body embodiment within the scope of the present invention.

FIG. 9 is a top plan view of a preferred elliptic cylindrical body embodiment within the scope of the present invention.

FIG. 10 is a top plan view of a nebulous cylindrical-like body embodiment within the scope of the present invention.

FIG. 11 is a top plan view of an elliptic cylindrical-like body embodiment within the scope of the present invention.

FIG. 12 is an exemplification of the steps of an embodiment of the current method of using the stabilizer for assisting the stabilization of a spinal implant.

FIG. 13 is a diagrammatic representation of the steps of another embodiment of the current method of using the stabilizer for assisting the stabilization of a spinal implant.

FIG. 14 is another diagrammatic representation of the steps of still another embodiment of the current method of using the stabilizer for assisting the stabilization of a spinal implant.

### DESCRIPTION OF THE PREFERRED EMBODIMENTS

Although the disclosure hereof is detailed to enable those skilled in the art to practice the invention, the embodiments published herein merely exemplify the present invention.

The practice of the present invention requires the removal of mammalian tissue to create a cavity for receiving a spinal implant. Depending on the surgical procedure performed, spinal implants of varying sizes and shapes may be selected for eventual implantation into the surgical cavity. Preferred embodiments of the current stabilizer are manufactured in differing sizes and shapes and are compatible with numerous spinal implants of amorphous or geometrically defined spinal supporting structures.

In the most general sense, the current invention is a stabilizer for assisting with the stabilization of a spinal implant that will be implanted into the surgically created cavity. Stabilizers in accord with the present invention can have polygonal, polygonal-like, cylindrical, cylindrical-like, elliptic cylindrical or elliptic cylindrical-like bodies. In other words, when engineering parameters require, the polygonal-like, cylindrical-like and elliptic cylindrical-like bodies can include one or more nebulous segments modifying the standardized geometric polygonal, cylindrical and elliptic cylindrical bodies. Thus, select preferred embodiments of the stabilizer can be engineered to have a socket for fitting a spinal implant of virtually any outward supporting structure dimension. Unless otherwise indicated, for the remainder of this specification: the term “polygonal” body can also refer to a polygonal-like body; the term “cylindrical” body can also refer to a cylindrical-like body; and the term “elliptic cylindrical” body can also refer to an elliptic cylindrical-like body.

For preferred embodiments, an anterior wall or extension of the body includes a plurality of apertures and extends beyond an outward edge of the polygonal, cylindrical or elliptic cylindrical body. Each stabilizer further includes a cover that has an aperture that can be aligned with one of the plurality of apertures positioned on the body's anterior wall or extension. Fasteners secure the cover to the polygonal, cylindrical or elliptic cylindrical body as well as bone. After attachment to the polygonal, cylindrical or elliptic cylindrical body, the cover is capable of preventing egress of fasteners securing the stabilizer to bone. Stabilizers of the current invention can be manufactured of biocompatible metals, plastics or combinations thereof, and preferred embodiments are manufactured of titanium, titanium alloys, stainless steel, non-resorbable and resorbable polymers. The current stabilizer meets the long felt but unfilled need of providing a stabilizer for use with a spinal implant that has a polygonal, cylindrical or elliptic cylindrical shape, where the stabilizer is attached to bone at first end of the stabilizer and has a socket at the second end for connecting to a lengthwise end of the spinal implant.

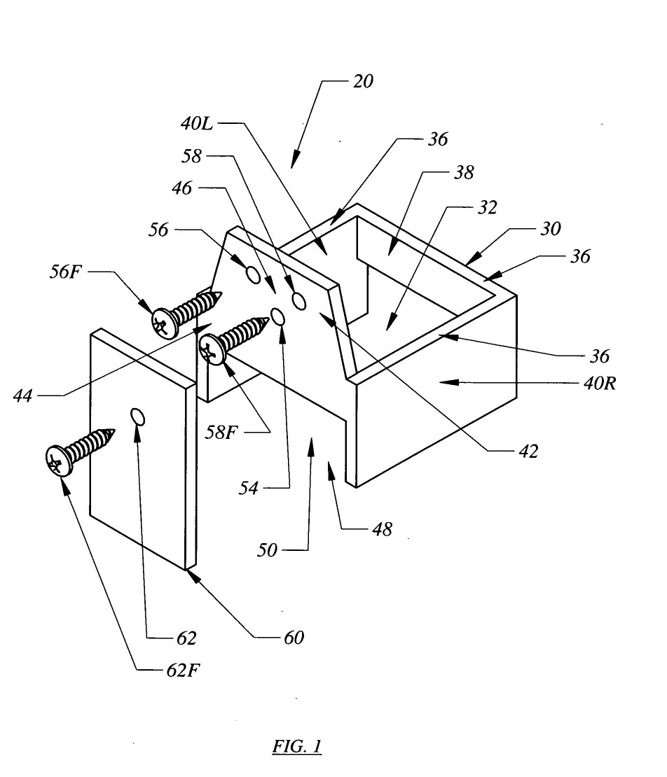

FIG. 1 is an exploded view frontal perspective of a preferred embodiment of the stabilizer (20) having a polygonal body (30) and attachable cover (60). Polygonal body (30) encloses opening or cavity (32). Perimeter (36) is distal from spinal implant (not shown in this view). Extending from perimeter (36) toward spinal implant are posterior wall (38) and lateral walls (40L and 40R). Posterior wall (38) and lateral walls (40L and 40R) extend approximately identical lengths from perimeter (36). Anterior wall (42) has first section (44) extending from perimeter (36) toward the spinal implant and second section (46) extending from perimeter (36) away from the spinal implant. Since first section (44) is of lesser length than posterior wall (38) and lateral walls (40L and 40R), socket (50) for fitting about a lengthwise end of the spinal implant is created. A lengthwise end of the spinal implant can be fitted through gap (48) into socket (50). Second section (46) of anterior wall (42) is provided with apertures (54, 56 and 58). In select preferred embodiments, a portion of anterior wall (42) extends beyond perimeter (36) in a direction away from the spinal implant. As shown in FIG. 1, perimeter (36) is open to cavity (32), but in select preferred embodiments perimeter (36) can be a continuous surface such that one end of cavity (32) is closed.

Cover (60) is provided with aperture (62) that upon attachment to polygonal body (30) aligns with aperture (54) of second section (46) of anterior wall (42). When cover (60) is attached to polygonal body (30), cover (60) prevents egress of fasteners (56F and 58F) from second section (46) of polygonal body (30). Fastener (62F) secures cover (60) at aperture (54) to second section (46) such that cover (60) blocks egress of the spinal implant (not shown in this view) from gap (48) and socket (50).

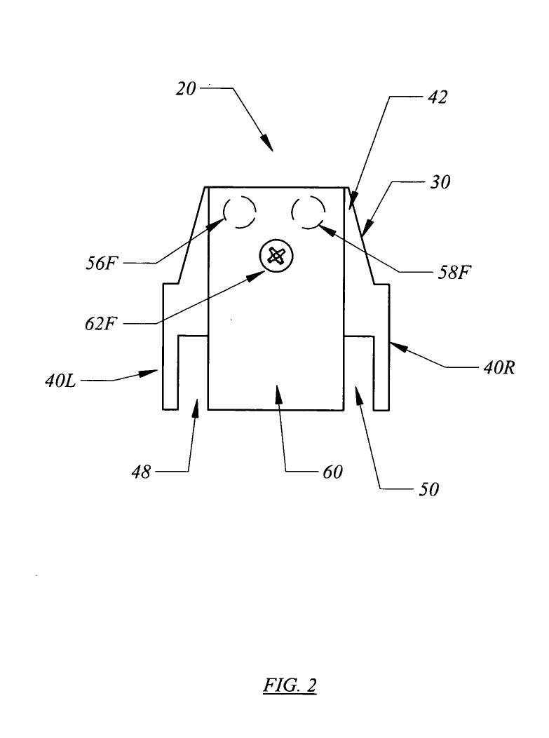

FIG. 2 is a frontal view of stabilizer (20) where cover (60) is attached to anterior wall (42) of polygonal body (30). Fastener (62) secures cover (60) to anterior wall to block egress of fasteners (56F and 58F) from apertures (56 and 58), respectively. Cover (60) is of sufficient length to block egress of the spinal implant (not shown in this view) from gap (48) and socket (50).

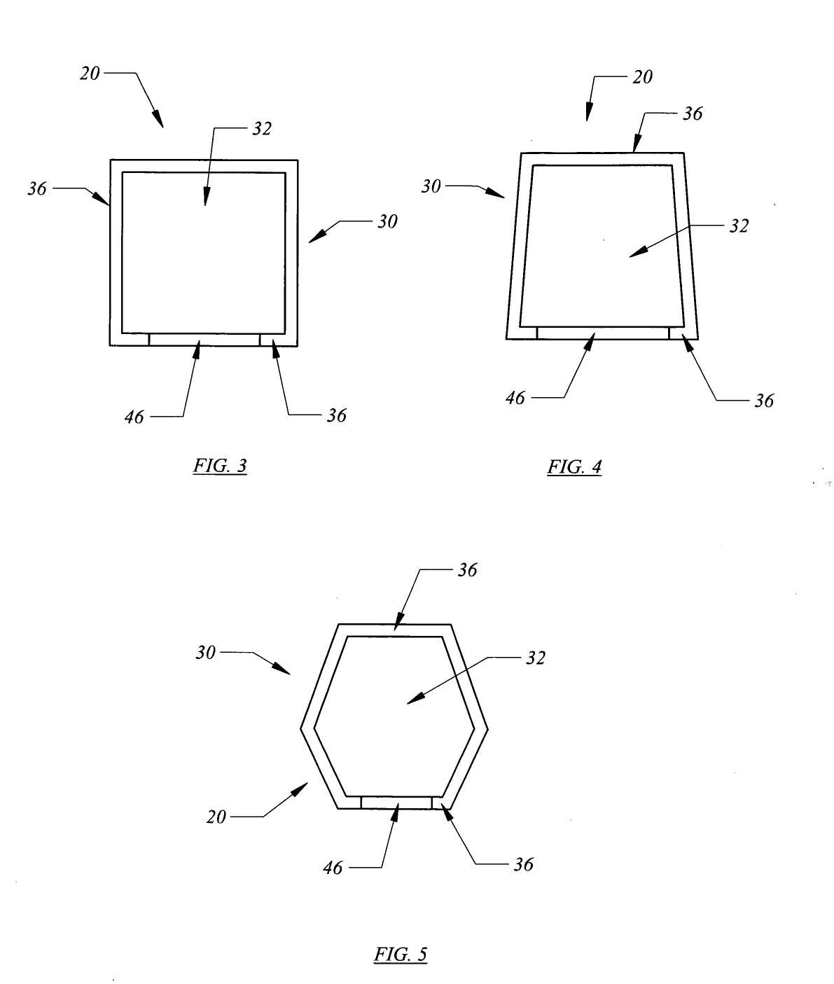

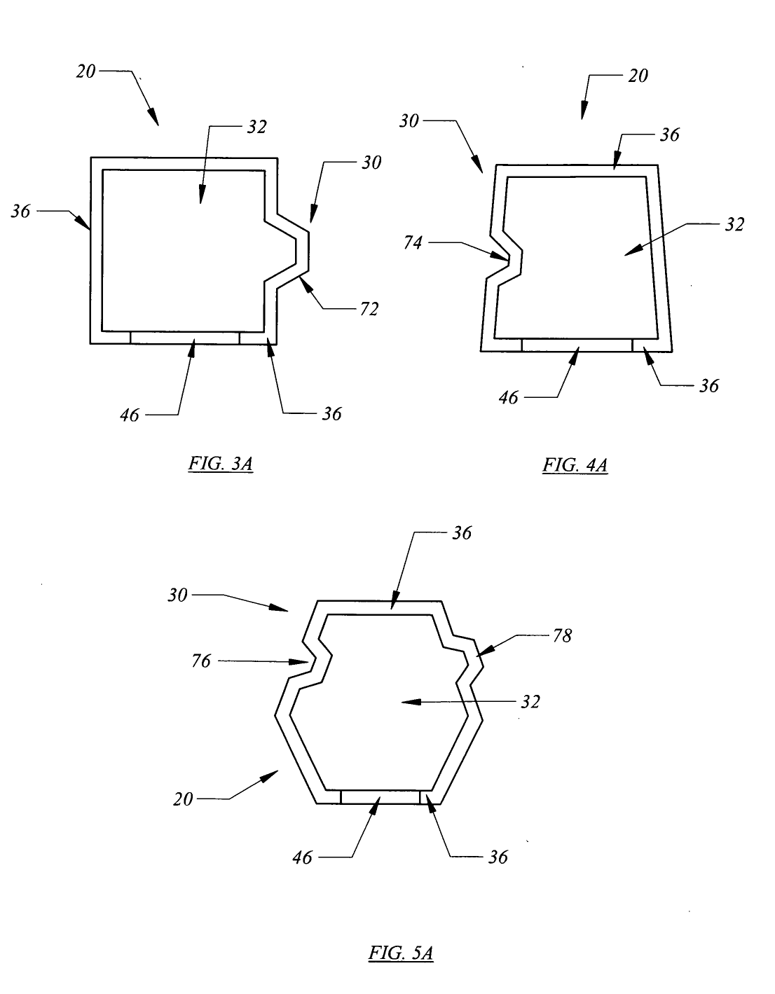

By way of illustration and not limitation, FIGS. 3-5 are top plan views of preferred embodiments of stabilizers (20) with different shaped polygonal bodies (30). FIG. 3 portrays a rectangular polygonal body (30). FIG. 4 shows a trapezoidal polygonal body (30). FIG. 5 enables a hexagonal polygonal body (30). Although not shown in FIGS. 3-5, depending on engineering parameters and the design of the spinal implant that will be fitted into the stabilizer's socket, polygonal stabilizers of other shapes are within the scope of the present invention.

In the preferred embodiments displayed in FIG. 3-5, openings or cavities (32), perimeters (36) and second sections (46) of anterior walls (42) are disclosed. For select preferred embodiments not shown in FIGS. 3-5, second sections (46) can be the depth of perimeter (36). In other words, second sections (46) of anterior walls (42) equate with the perimeters for depth and width of second sections (46).

By way of illustration and not limitation, FIGS. 3A-5A are top plan views of preferred embodiments of stabilizers (20) with amorphous or nebulous shaped polygonal-like bodies (30). FIG. 3A portrays one of a plethora of nebulous rectangular-like polygonal bodies (30) having amorphous segment (72). FIG. 4A shows one of many potential nebulous trapezoidal-like polygonal bodies (30) including amorphous segment (74). FIG. 5A enables one or a plethora of hexagonal-like polygonal bodies (30) having amorphous segments (76 and 78).

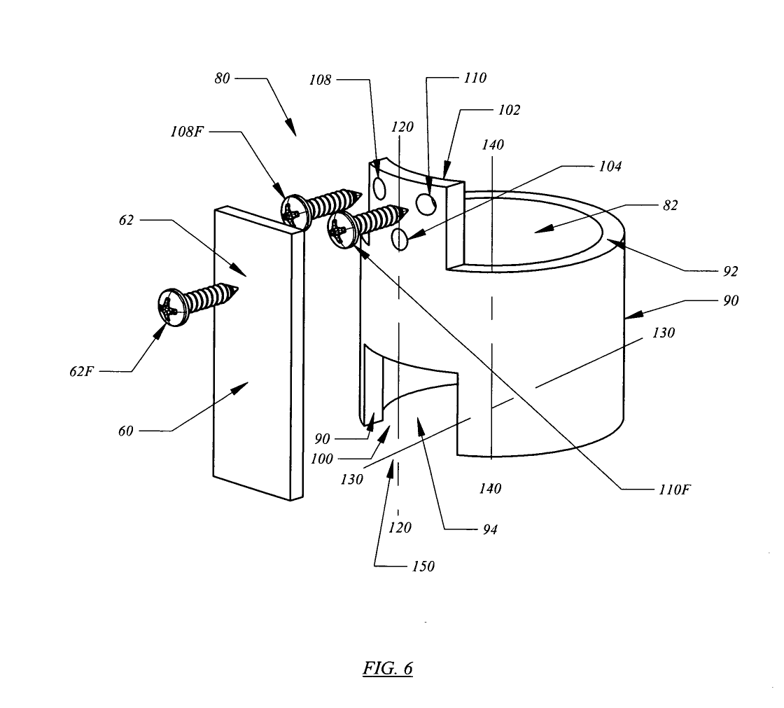

FIG. 6 portrays a preferred embodiment of a cylindrical or elliptic cylindrical stabilizer (80). Cylindrical or elliptic cylindrical wall (90) encloses lumen or cavity (82). Rim (92) is distal from the spinal implant (not shown in this view). Gap (94) of cylindrical or elliptic cylindrical wall (90) is distal from rim (92). Gap (94) in cylindrical or elliptic cylindrical wall (90) creates socket (100) for fitting about a lengthwise end of the spinal implant.

Extension (102) extends beyond rim (92) in a direction away from the spinal implant. In select preferred embodiments, extension (102) is proximate gap (94). Extension (102) is provided with apertures (104, 108 and 110). In select preferred embodiments, extension (102) follows the contour of rim (92). In still other select preferred embodiments, extension (102) is coplanar with central lengthwise axis (120-120) that intersects center (150) of gap (94) and is simultaneously perpendicular to axis (130-130) that is simultaneously perpendicular to central longitudinal axis (140-140) wherein axis (130-130) intersects center (150) of gap (94). As shown in FIG. 6, cavity (82) is open to rim (92), but in other select preferred embodiments a continuous surface covers cavity (82) to create an end of stabilizer (80) that is enclosed rather than open.

Cover (60) is provided with aperture (62) that upon attachment to extension (102) of stabilizer (80) corresponds to aperture (104) of extension (102). When cover (60) is attached to stabilizer (80), cover (60) prevents egress of fasteners (108F and 110F) from extension (102) of stabilizer (80). Fastener (62F) secures cover (60) at aperture (104) of extension (102) such that cover (60) blocks egress of the spinal implant (not shown in this view) from gap (94) and socket (100).

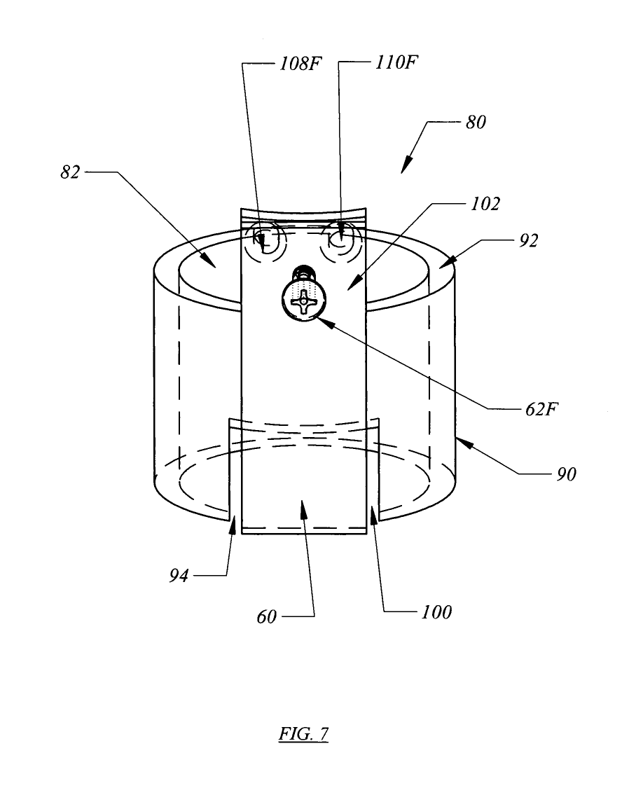

FIG. 7 is a frontal perspective of stabilizer (80) where cover (60) is attached to extension (102). Fastener (62) secures cover (60) to stabilizer (80) to block egress of fasteners (108F and 110F) from apertures (108 and 110), respectively. Cover (60) is of sufficient length to block egress of the spinal implant (not shown in this view) from gap (94) and socket (100).

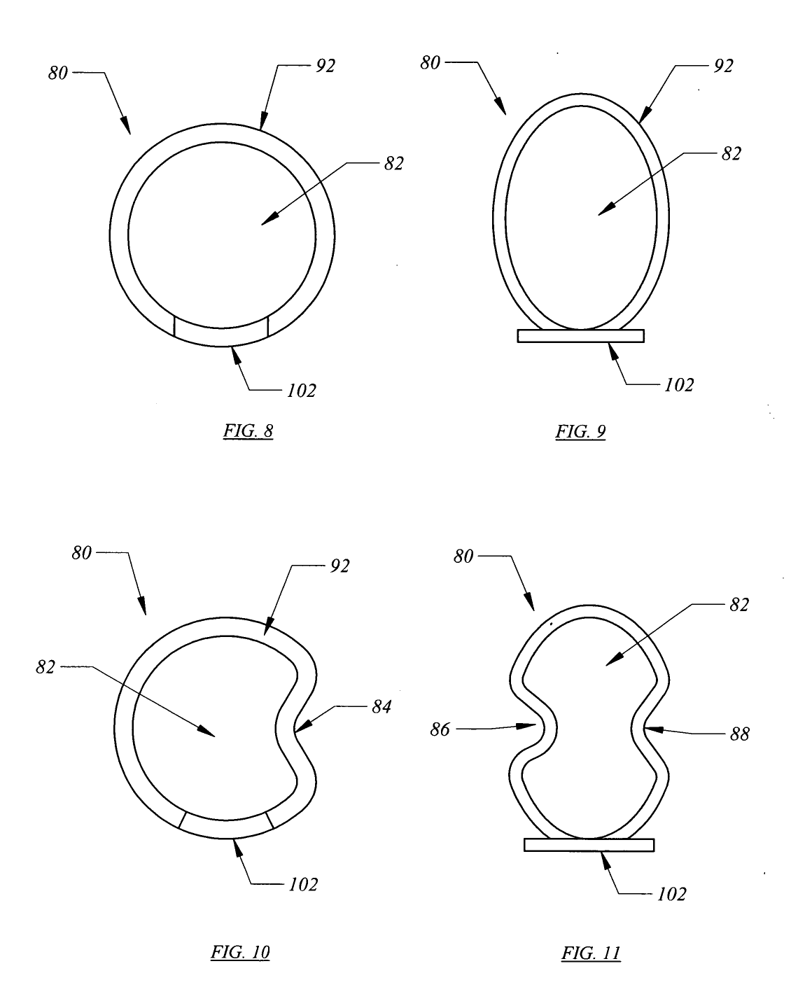

By way of illustration and not limitation, FIGS. 8-11 are top plan views of preferred embodiments of cylindrical or elliptic cylindrical stabilizer (80). FIG. 8 portrays cylindrical body (80) including lumen (82), rim (92) and extension (102). FIG. 9 shows an elliptic cylindrical body (80) having lumen (82), rim (92) and extension (102). FIG. 10 enables one of a plethora of nebulous cylindrical-like bodies (80) having lumen (82), amorphous segment (84), rim (92) and extension (102). FIG. 11 shows one of a plethora of nebulous elliptic cylindrical-like bodies including lumen (82), amorphous segments (86 and 88), rim (92) and extension (102).

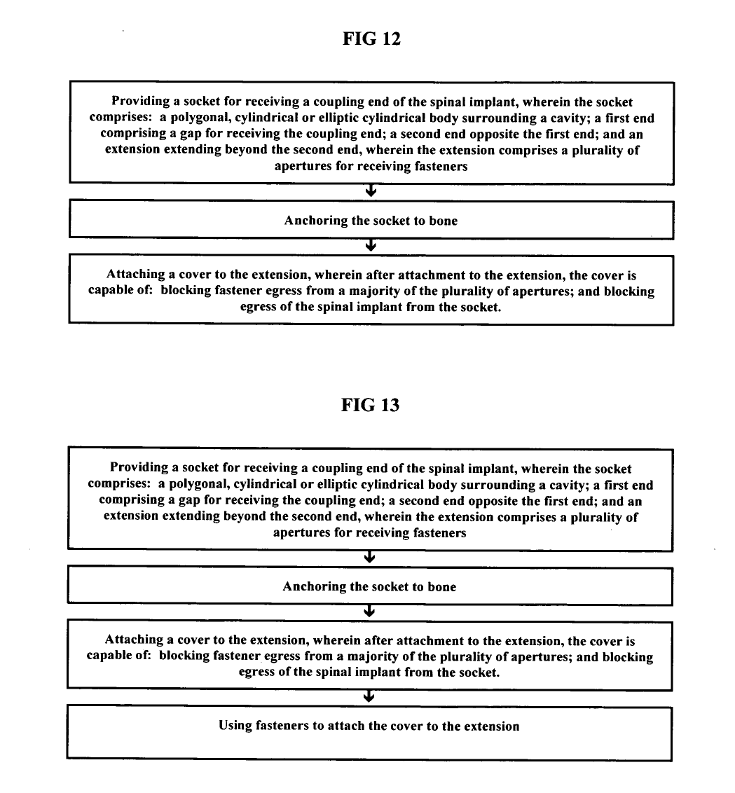

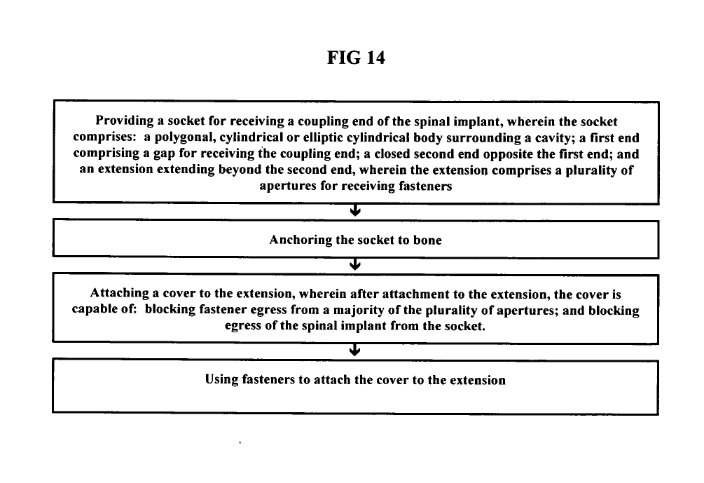

Steps associated with the practice of the methods of embodiments the present invention are set forth in FIGS. 12-14. Those steps are related to the practice of using the stabilizer structures previously set forth.

Having disclosed the invention as required by Title 35 of the United States Code, Applicant now prays respectfully that Letters Patent be granted for his invention in accordance with the scope of the claims appended hereto.

What is claimed is:

1) A stabilizer for assisting stabilization of a spinal implant; said stabilizer comprising:
a) a polygonal body comprising:
i) a perimeter distal from said spinal implant enclosing an opening of said polygonal body;
ii) posterior and lateral walls extending from said perimeter toward said spinal implant, wherein said posterior and lateral walls are of approximately identical length; and
iii) an anterior wall comprising a first section extending from said perimeter toward said spinal implant and a second section extending from said perimeter away from said spinal implant, wherein:
A) said first section is of lesser length than said posterior and lateral walls, thereby creating a socket for fitting about a lengthwise end of said spinal implant; and
B) said second section comprises a plurality of apertures for receiving fasteners;
b) a cover attachable to said second section capable of:
i) blocking fastener egress from a majority of said plurality of apertures; and
ii) blocking egress of said spinal implant from said socket; and
c) one or more fasteners for securing said cover to said second section.

2) The invention of claim 1, wherein said polygonal body further comprises a closed end.

3) The invention of claim 1, wherein combination of said perimeter and said walls of said polygonal body creates a polygonal body that is generally rectangular.

4) The invention of claim 3, wherein said polygonal body further comprises an amorphous segment.

5) The invention of claim 1, wherein combination of said perimeter and said walls of said polygonal body creates a polygonal body that is generally trapezoidal.

6) The invention of claim 5, wherein said polygonal body further comprises an amorphous segment.

7) The invention of claim 1, wherein combination of said perimeter and said walls of said polygonal body creates a polygonal body that is generally hexagonal.

8) The invention of claim 7, wherein said polygonal body further comprises an amorphous segment.

9) A stabilizer for assisting stabilization of a spinal implant; said stabilizer comprising:
a) a cylindrical or an elliptic cylindrical wall encircling a lumen;
b) an opening at each lengthwise end of said wall;
c) a rim distal from said spinal implant;
d) a gap in said wall at an end of said wall opposite said distal rim creating a socket for fitting about said lengthwise end of said spinal implant;
e) an extension proximate said gap and extending beyond said distal rim in a direction away from said spinal implant; said extension comprising a plurality of apertures for receiving fasteners;
f) a cover, including a plurality of apertures, attachable to said extension and capable of:
i) blocking fastener egress from a majority of said plurality of apertures; and
ii) blocking egress of said spinal implant from said socket; and
g) one or more fasteners for securing said cover to said second section.

10) The invention of claim 9, wherein said cylindrical or said elliptic cylindrical wall further comprises a closed end.

11) The invention of claim 10, wherein said wall further comprises an amorphous segment.

12) A socket for assisting stabilization of a spinal implant; said socket comprising:
a) a polygonal, cylindrical or elliptic cylindrical body surrounding a cavity; said body comprising:
i) a first end for coupling with an end of said spinal implant, wherein said first end further comprises a gap for receiving said coupling end of said spinal implant into said cavity of said socket;
ii) a second end opposite said first end; and
iii) an extension extending beyond said second end, wherein said extension comprises a plurality of apertures for receiving fasteners; and
b) a cover attachable to said extension capable of:
i) blocking fastener egress from a majority of said plurality of apertures; and
ii) blocking egress of said spinal implant from said socket.

13) The invention of claim 12 further comprising one or more fasteners.

14) The invention of claim 13, wherein said polygonal, cylindrical or elliptic cylindrical body comprises an amorphous segment.

15) The invention of claim 14, wherein said second end is closed.

16) A method of assisting with stabilization of a spinal implant comprising the steps of:
a) providing a socket for receiving a coupling end of said spinal implant, wherein said socket comprises:
i) a polygonal, cylindrical or elliptic cylindrical body surrounding a cavity;
ii) a first end comprising a gap for receiving said coupling end;
iii) a second end opposite said first end; and
iv) an extension extending beyond said second end, wherein said extension comprises a plurality of apertures for receiving fasteners;
b) anchoring said socket to bone; and
c) attaching a cover to said extension, wherein after attachment to said extension, said cover is capable of:
i) blocking fastener egress from a majority of said plurality of apertures; and
ii) blocking egress of said spinal implant from said socket.

17) The method of claim 16 further comprising the step of using fasteners to attach said cover to said extension.

18) The method of claim 17 further comprising the step of enclosing an end of said socket.

19) The method of claim 17 further comprising the step of altering said body to include an amorphous segment.

20) The method of claim 17 further comprising the step of enclosing an end of said socket.
